(19) United States
(12) Patent Application Publication
(10) Pub. No.: US 2011/0190455 A1
(43) Pub. Date: Aug. 4, 2011

(54) POLYDIALKYLSILOXANE-BRIDGED BI-PHOTOCHROMIC MOLECULES

(75) Inventor: Steven Michael Partington, West Yorkshire (GB)

(73) Assignee: Vivimed Labs Europe Ltd., Huddersfield (GB)

(21) Appl. No.: 12/737,795

(22) PCT Filed: Aug. 18, 2009
(86) PCT No.: PCT/GB2009/002010

§ 371 Date: Apr. 20, 2011

(30) Foreign Application Priority Data
Aug. 18, 2008 (EP) 0815109.4

(51) Int. Cl. C08F 283/12; C07D 311/02; C07F 7/08

(52) U.S. Cl. 525/418; 549/214; 544/69

(57) ABSTRACT

A bi-photochromic molecule comprises two photochromic moieties linked via a polydialkylsiloxane oligomer. An ophthalmic lens comprises the bi-photochromic molecule. A polymeric host material comprises the bi-photochromic molecule.

The present invention relates to photochromic molecules, in particular bi-photochromic molecules comprising a polydialkylsiloxane oligomer linker, and to products comprising them.

Photochromism is a well known physical phenomenon, which is defined as “a reversible transformation of a single chemical species being induced in one or both directions by electromagnetic radiation between two states having different distinguishable absorption spectra”. A detailed discussion of this phenomenon can be found in “Photochromism: Molecules and Systems”, revised edition, edited by H. Durr and H. Bouas-Laurent, Elsevier, 2003. A review of the major classes of organic photochromic molecules can be found in “Organic Photochromic and Thermochromic Compounds, Volume 1, Main Photochromic Families”, edited by J. Grano and R. Guglielmetti, Plenum Press, 1999. A detailed review of photochromic naphthopyrans can be found in “Functional Dyes”, edited by Sung-Hoon Kim, pages 85-137, Elsevier, Amsterdam, 2006.

Currently the major business area for photochromic molecules is the ophthalmic market, where T-type (thermally reversible) photochromics are used. The most important classes of organic photochromic molecules for the ophthalmic market are the naphthopyrans (both the 1,2-b and 2,1-b ring systems), and the spiro-naphthoxazines (both the 1,2-b and 2,1-b ring systems). This has been an area of considerable patent activity, for example U.S. Pat. No. 5,650,098 (1,2-b naphthopyrans, Transitions), U.S. Pat. No. 5,623,005 (2,1-b naphthopyrans, Pilkington), U.S. Pat. No. 5,446,151 (2,1-b naphthoxazines, Pilkington), and U.S. Pat. No. 6,303,673 (1,2-b naphthoxazines, James Robinson).

Work has been carried out to alter the photochromic properties and the physical properties of the photochromic molecules, in an attempt to “tune” the properties of the molecule to those required by particular applications. One approach has been to attach various long chain substituents. Enichem (EP 0524692) claim oxazines with long chain alkoxy substituents and long chain ester substituents.

A patent from Polymers Australia (WO 04/41961), reveals the effects of polydimethylsiloxane chains, perfluoroalkyl chains, polyethylene glycol chains, and alkyl chains on the fade speeds of single photochromic molecules in rigid polymeric matrices of high glass transition temperature (T_{g}). This patent reveals that the greatest increase in photochromic fade speeds of single photochromic molecules was caused by polydimethylsiloxane chains. Subsequent patents from Polymers Australia reveal the effects of polymethyl (methacrylate) and polybutacrylate chains generated by “living polymerisation” (WO 05/105875, WO 06/24099), and polyether chains (WO 05/105874). A literature article from the authors of the Polymers Australia patents (R. Evans et al, Nature 2005, Vol 4, p 249) indicates that use of polydimethylsiloxane chains gave the greatest improvements in increasing the rate of fade of single photochromic compounds in an ophthalmic lens matrix. Commercially an increased rate of fade, whilst still achieving an acceptable intensity of colour, is a desirable property for ophthalmic lenses.

Polymerisable groups have been attached to oxazines (U.S. Pat. No. 5,821,287, National Science Council Taiwan). Polymerisable polyalkoxylated pyrans have been claimed by PPG (WO 00/15629) and Transitions (WO 03/56390).

Work has also been carried out to link two photochromic units by means of a bridge. Guglielmetti et al have linked oxazines and pyrans by means of ethane, ethylenic, acetylenic, ester, mono-, bi- and ter-thiophene bridges (see F. Ortica et al, J. Photochem. Photobiol A, (2001), 139, 2-3; M. Frigoli et al, Helv. Chim. Acta, Vol 83, (2000), P 3043-3052; A. Yassar et al, Applied Physics Letters, (2002), Vol 80, 23, P 4297-4299). Rodenstock (EP 0686685) have linked pyrans by means of a —CH₂CH₂— bridge which, it is taught, does not affect the photochromic properties of the photochromic moieties; it is therefore clear that this bridge does not give any advantages in terms of improved properties such as fade rate or colour intensity. Zhao and Carreira (JACS 2002, 124, 8, p 1582) have prepared bis-naphthopyrans linked by a bis-thiophene, by phenyl groups (Organic Letters, 2006, Vol 8 No. 1, p 99) and by oligothiophenes (Chem. Eur. J. 2007, 13, 2671-2685), Coelho et al (Tetrahedron, 2005, 61, p 11730) have linked pyrans by means of phenyl, phenyl-O -phenyl, and phenyl-CH₂CH₂-phenyl bridges. Great Lakes (WO 00/39245) claim a trimeric species where three oxazines are attached to a central triazine. Great Lakes (WO 00/05325 and WO 00/21968) also claim compounds where two, three or four oxazines are linked to a central tetramethylcyclotetrasiloxane ring.

However, many of these known molecules suffer from disadvantages including slow fade rates, poor colour strength, and poor heat stability. As a result, many of these molecules are not well-suited for certain uses such as, for example, incorporation into ophthalmic lenses. There exists, therefore, a need for photochromic molecules exhibiting improved properties.

According to the present invention in its broadest aspect, there is provided a bi-photochromic molecule comprising two photochromic moieties linked via a polydialkylsiloxane oligomer.

In a further aspect, there is provided a bi-photochromic molecule having the structure set out in claim 4.

There is also provided an ophthalmic lens comprising a bi-photochromic molecule according to the invention.

In a further aspect, the invention provides a polymeric host material comprising a bi-photochromic molecule according to the invention.

It has been found that the molecules exhibit a considerable improvement in the rate of fade in polymer matrices compared to the parent photochromic molecules. The compounds of the invention often exhibit increased strength of photochromic colour compared to the parent photochromic molecule, allowing for molecular weight and the number of photochromic units present. The compounds of the invention are particularly useful for use in photochromic ophthalmic lenses.

It has also unexpectedly been found that these molecules have improved heat stability when incorporated into polymers, compared to the individual photochromic molecules which are not linked by the bridging group. This allows the molecules of the invention to be incorporated into polymers which require higher processing temperatures than are compatible with the unlinked photochromic molecules.

The molecules of this invention also have the beneficial property of a lower yellowness index compared to the individual photochromic molecules which are not linked via a polydialkylsiloxane oligomer when processed at the same temperature in the same polymer.

Similarly, we believe that the compounds of the invention have advantages of improved fade rate, improved photochromic colour strength, increased heat stability and reduced yellowness index when compared to the known bi-photochromic compounds comprising bridging groups.

In a preferred embodiment of the present invention, two photochromic molecules are linked by means of a bridge which comprises a linking group at each end of a central polydialkylsiloxane (PDAS) chain to provide novel polydialkylsiloxane bridged bi-photochromic molecules. Preferably, the bridge consists of a linking group at each end of a central PDAS chain.

The photochromic units may be the same or different, allowing for the possibility of different chromophores with different fade rates to be present in the same molecule.
The molecules of the invention comprise two photochromic moieties or molecules linked via a polydialkylsiloxane chain. It is highly preferred that the polydialkylsiloxane bridge, or linker, comprises a linking group at each end. Preferably, the bridge, or linker, consists of a linking group at each end of a central polydialkylsiloxane chain. Any suitable polydialkylsiloxane chain and linking groups may be employed.
Preferably, the compounds are of the general formula:

PC-L-PDAS-L′-PC′
wherein PC and PC′ represent a photochromic moiety; PDAS represents a polydialkylsiloxane chain; and L and L′ represent linking groups.
PC and PC′ may be the same or different. It is particularly preferred that PC and PC′ independently represent photochromic moieties of general structure I to IV:

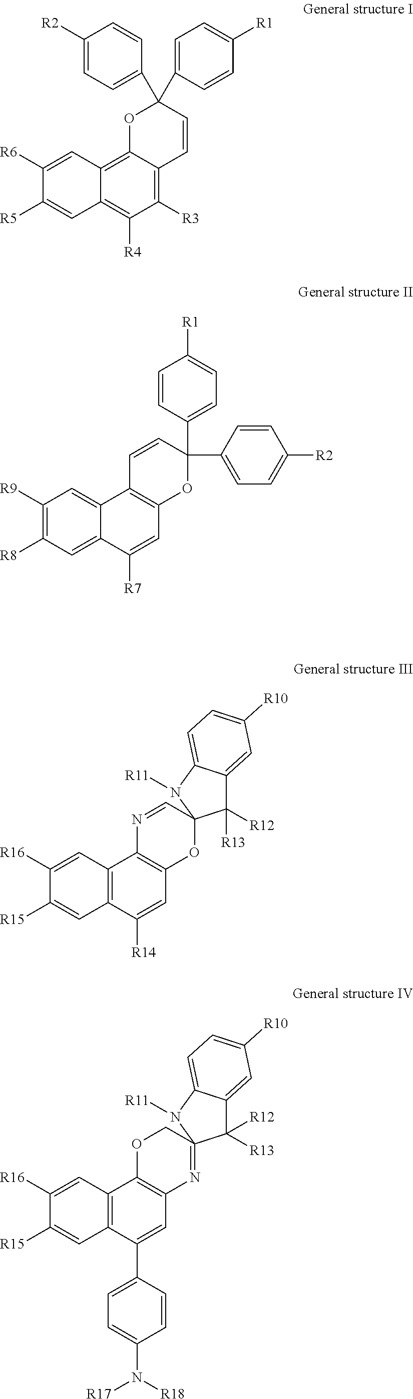

wherein R1 and R2 independently represent hydrogen, linear or branched C₁₋₁₀ alkyl, linear or branched C₁₋₁₀ alkoxy, C₁₋₁₀ hydroxyalkoxy, C₁₋₁₀ alkoxy(C₁₋₁₀)alkoxy, phenyl, C₁₋₁₀ alkoxyphenyl, halogen, C₁₋₅ haloalkyl, C₁₋₅ alkylamino, C₁₋₅ dialkylamino, arylamino, diarylamino, aryl C₁₋₅ alkylamino, or a cyclic amino group;
R3 represents hydrogen, linear or branched C₁₋₁₀ alkyl, C₃-C₂₀ cycloalkyl, C₅-C₂₀ bicycloalkyl, linear or branched C₂₋₁₀ alkenyl, linear or branched C₁₋₁₀ alkoxy, C₁₋₁₀ hydroxyalkyl, C₁₋₁₀ aminoalkyl, linear or branched C₁₋₂₀ alkoxycarbonyl, carboxyl, halogen, aryloxycarbonyl, formyl, acetyl or aroyl;
R4 represents phenyl, C₁₋₁₀ alkoxyphenyl, C₁₋₁₀ dialkoxyphenyl, C₁₋₁₀ alkylphenyl, C₁₋₁₀ dialkylphenyl, in addition to those groups specified for R3;
or R3 and R4 together form a cyclic structure of the type

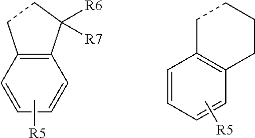

R5, R6, R7, R8, R9, R10, R14, R15, R16 are as defined above for R1 and R2;
R11 represents linear or branched C₁₋₂₀ alkyl, C₃-C₂₀ cycloalkyl, C₆-C₂₀ bicycloalkyl, (C₁₋₅ alkyl)aryl, (C₁₋₅ alkyl)cycloalkyl, (C₁₋₅ alkyl)bicycloalkyl, C₁₋₅ haloalkyl, C₁₋₅ dihaloalkyl or C₁₋₅ trihaloalkyl;
R12 and R13 represent C₁₋₁₀ alkyl, C₁₋₅ alkyl alkoxycarbonyl, or together form a C₅₋₇ ring; and
R17 and R18 represent linear or branched C₁₋₁₀ alkyl, C₁₋₁₀ hydroxyalkyl, or together form a C₅₋₇ ring.

L and L′, which may be the same or different, represent a linking group. Any suitable linking group may be used. It is preferred that L and L′ represent a linking group of the form

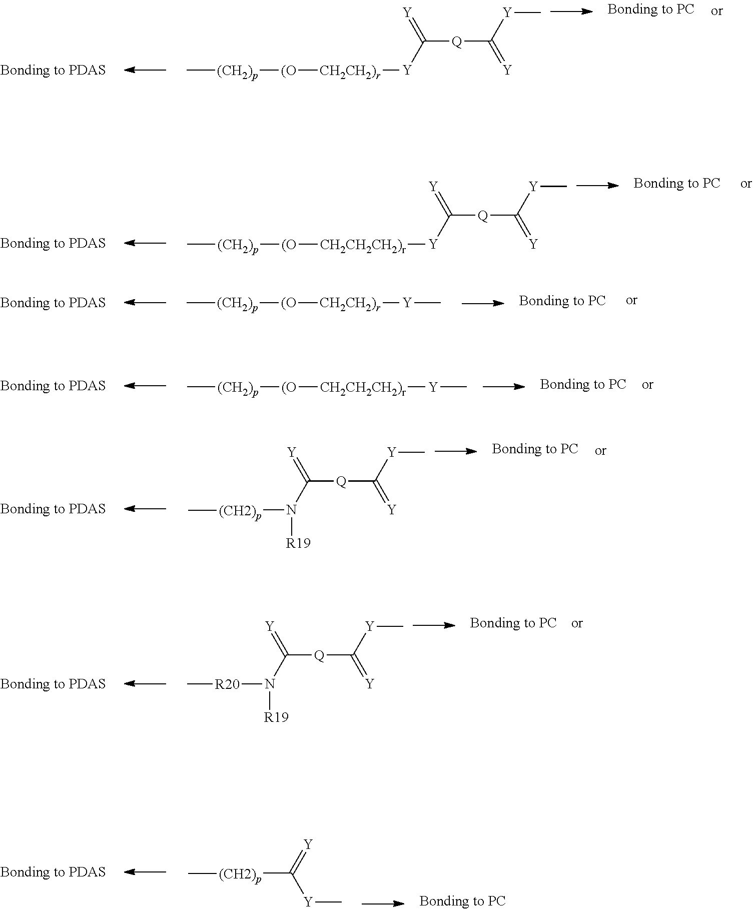

wherein Y is independently oxygen or sulphur, R19 is hydrogen or C₁₋₁₀ linear or branched alkyl, R20 is C₁₋₁₀ linear or branched alkyl, p is an integer from 1 to 15, and r is an integer from 0 to 10, and wherein Q is linear or branched C₁₋₁₀ alkyl, C₁₋₁₀ alkenyl or 1,2-, 1,3, or 1,4-substituted aryl, or substituted heteroaryl.

Preferably Y is oxygen.

Particularly preferred linker groups L and L′ are:

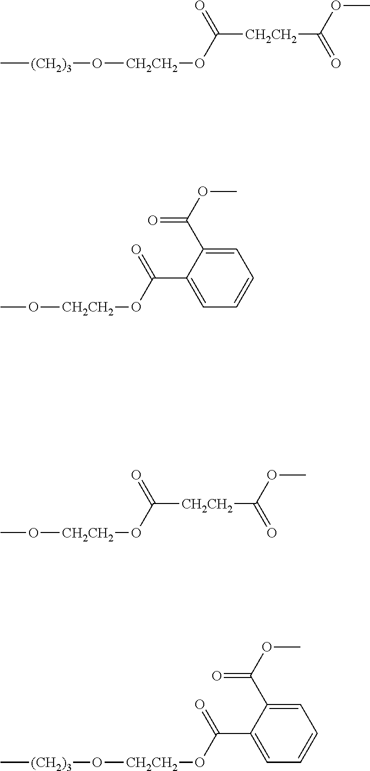

PDAS represents a polydialkylsiloxane chain. Preferably, PDAS represents an oligomer of the form

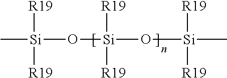

wherein R19 is C₁₋₁₀ alkyl, and n is an integer of from 4 to 75.

Polydialkylsiloxane oligomers are commercially available, for example from Gelest Inc, Shin-Etsu Chemical Co. Ltd; Chisso Corp; Toshiba Silicone Co. Ltd; and Toray-Dow Corning Co. Ltd.

Suitable polydialkylsiloxane oligomers include, but are not limited to, polydimethylsiloxane oligomers, such that R19 is preferably methyl.

It is preferred that n is between 6 and 30 inclusively. Particularly preferably, R19 is methyl and n is an integer of from 6 to 30.

Preferred polydimethylsiloxane oligomers include the oligomers DMS-B12, DMS-C15, DMS-C16, DMS-C21, DMS-A11, DMS-A12, DMS-A15, DMS-A21, DMS-A211 and DMS-A214 available from Gelest Inc; KF-6001, KF-6002, KF-6003, KF-8010, X-22-160AS, X-22-162A, X-22-161A, X-22-161B and X-22-162C from Shin-Etsu; and Silaplane FM-44 from Chisso. Particularly preferred are oligomers DMS-B12, DMS-C15, DMS-C16, DMS-C21, DMS-A11, DMS-A12, DMS-A15, DMS-A21, DMS-A211 and DMS-A214 from Gelest. These are quoted as having the following structures and approximate molecular weight or molecular weight ranges. For convenience, the Gelest nomenclature is used to name the following polydimethylsiloxane oligomers, rather than the cumbersome (and not strictly accurate, as the oligomers are mixtures) systematic names.

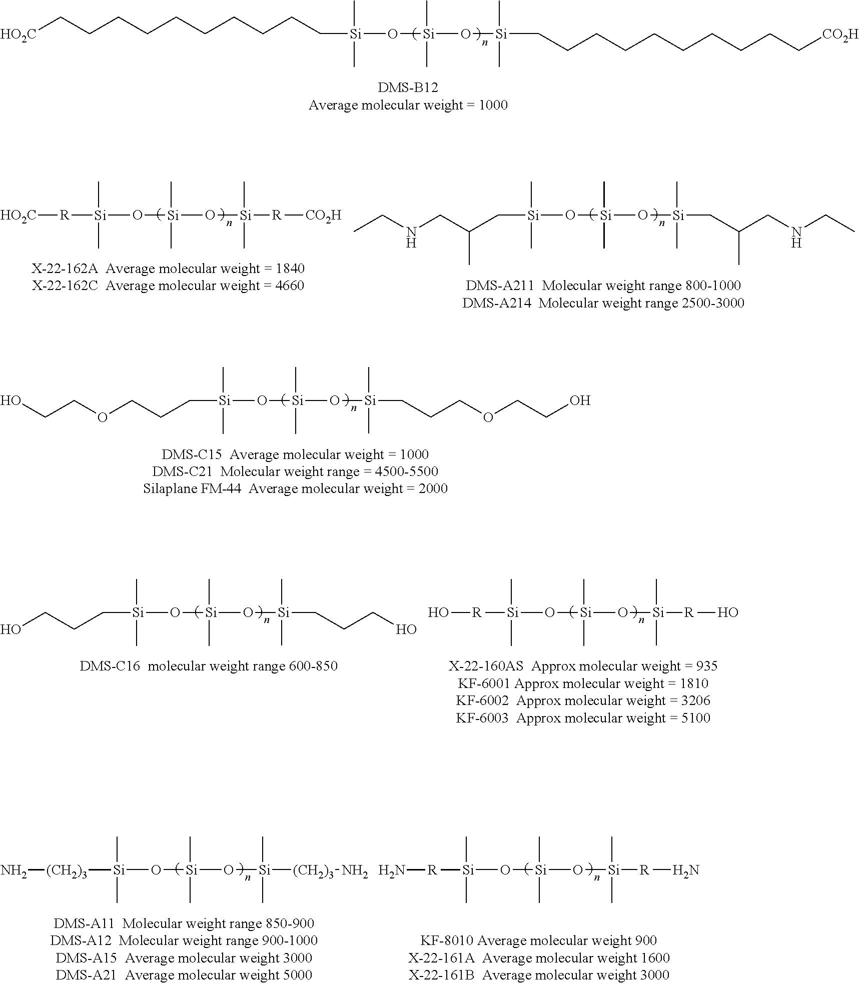

As the skilled person is aware, commercially available polydimethylsiloxane oligomers are generally supplied either with an average molecular weight or a molecular weight range, and any number quoted as the number of repeat units of the dimethylsiloxane is to be interpreted as an average value.

The parent photochromic compounds may be prepared as described in U.S. Pat. No. 5,650,098 (1,2-b naphthopyrans), U.S. Pat. No. 5,623,005 (2,1-b naphthopyrans), U.S. Pat. No. 5,446,151 (2,1-b naphthoxazines), and U.S. Pat. No. 6,303,673 (1,2-b naphthoxazines).

Typically a linking group is attached to the commercially available oligomer, if required, and this reagent is then reacted with the parent photochromic compound to give the polydialkylsiloxane-bridged bi-photochromic molecule. The linking group may also be attached to the parent photochromic compound, which is then reacted with the commercially available oligomer to give the polydialkylsiloxane-bridged bi-photochromic molecule. Suitable reaction conditions will be apparent to the skilled person.

The following examples serve to illustrate the invention, and do not limit its scope.

### EXAMPLES

Commercially available polydimethylsiloxane oligomers are supplied either with an average molecular weight or a molecular weight range, and any number quoted as the number of repeat units of the dimethylsiloxane is to be interpreted as an average value. Accordingly, any yields quoted in the following Examples are inevitably approximate. The oligomers DMS-B12, DMS-C15, DMS-C16 and DMS-A214 are available from Gelest Inc. and are quoted as having the following structures and approximate molecular weight or molecular weight ranges. The Gelest nomenclature will be used to name the polydimethylsiloxane oligomer section of the polydialkylsiloxane-bridged bi-photochromic molecules, rather than the cumbersome (and not strictly accurate, as the oligomers are mixtures) systematic names. For yield calculations with DMS-C16 and DMS-A214, the midpoint of the molecular weight range has been used.

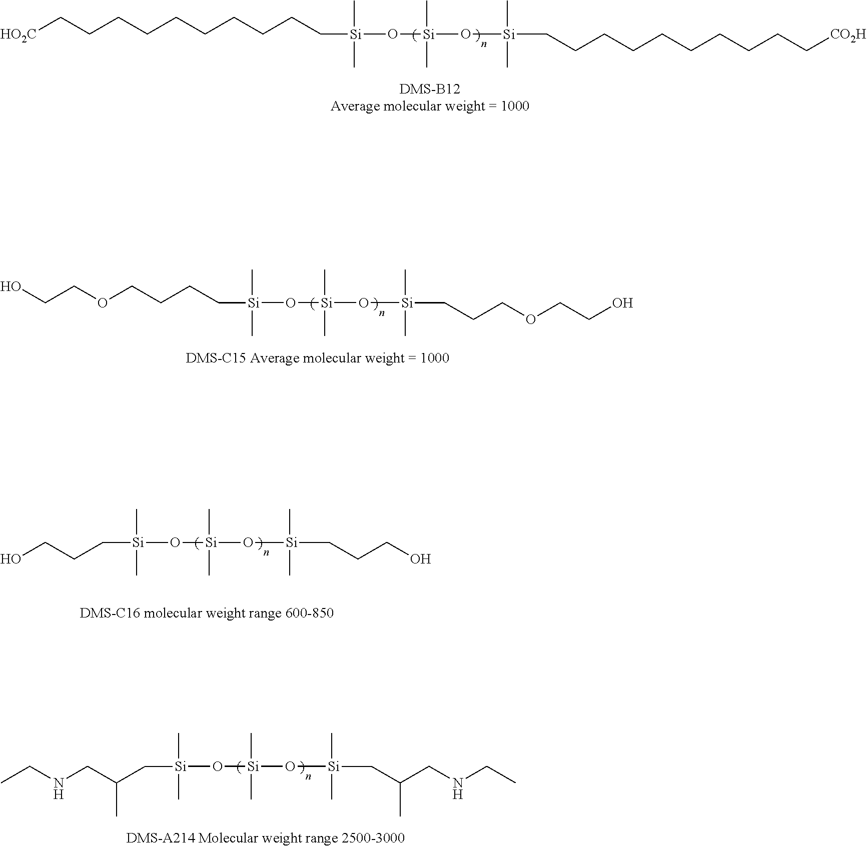

### Example 1

### Bis-succinyl-DMS-C15

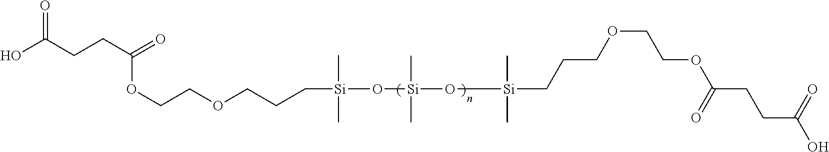

The bis-hydroxy-terminated siloxane DMS-C15 (9.1 g, molecular weight=1000) was mixed with succinic anhydride (2.8 g) and toluene (120 ml) for 2 minutes. Triethylamine (5.0 ml=3.5 g) was added and the mixture was heated to 70-75° C. for 1.5 hours. The solution was cooled to 25° C., then PEG monomethylether (2.6 g) was added and the mixture stirred for 20 minutes.

The solution was washed twice with a mixture of HCl (5 ml) and water (100 ml), then was washed with saturated brine (3×100 ml). The organic layer was dried over sodium sulphate, and filtered to give 110.2 g. Theoretical yield=10.9 g, giving a maximum strength of 9.9%.

### Example 2

### Bis-phthaloyl DMS-C15

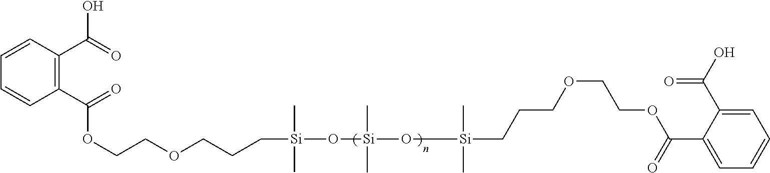

The reagent bis-phthaloyl-DMS-C15 was prepared in analogous fashion to bis-succinyl-DMS-C15 in Example 1, using an equivalent quantity of phthalic anhydride in place of succinic anhydride.

### Example 3

### Bis-succinyl-DMS-C16

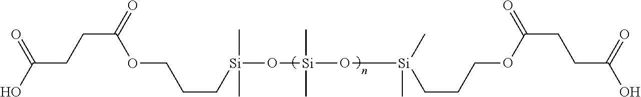

The reagent bis-succinyl-DMS-C16 was prepared in analogous fashion to bis-succinyl-DMS-C15 (Example 1), using the bis-hydroxy-terminated polydimethylsiloxane DMS-C16 (molecular weight range=600-850).

### Example 4

### Bis-succinamido-DMS-A214

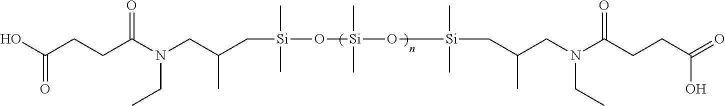

The reagent bis-succinamido-DMS-A214 was prepared in analogous fashion to bis-succinyl-DMS-C15, using the bis-secondary amino-terminated polydimethylsiloxane DMS-A214 (molecular weight range=2500-3000).

### Example 5

### (1,3-Dihydro-3,3-dimethyl-1-neopentyl-6′-(4″-N-ethyl, N-succinylethyl)anilino)spiro[2H-indole-2,3′-3H-naphtho[1,2-b][1,4]oxazine)₂-DMS-C15

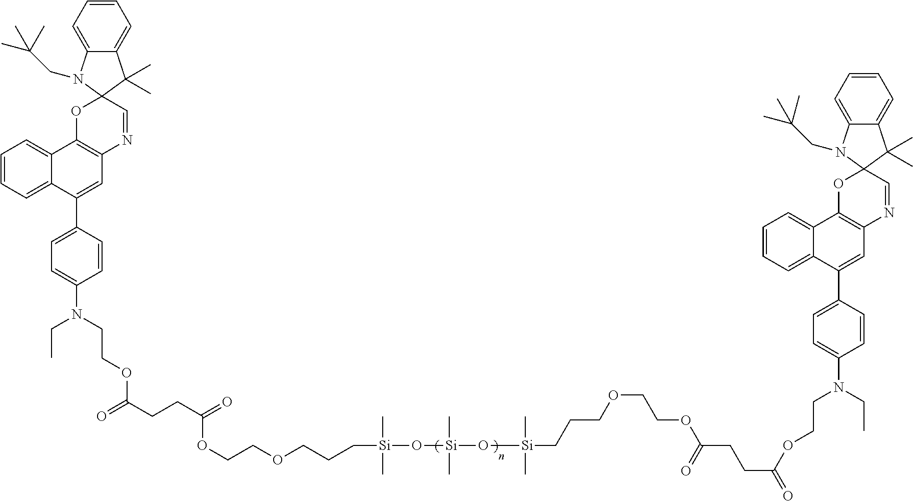

1,3-Dihydro-3,3-dimethyl-1-neopentyl-6′-(4″-N-ethyl, N-hydroxyethylanilino)spiro[2H-indole-2,3′-3H-naphtho[1,2-b][1,4]oxazine] (1.00 g) was mixed with a toluene solution of bis-succinyl-DMS-C15 (prepared according to Example 1, 18.5 g at 6.4% in toluene=1.18 g at 100%), dimethylaminopyridine (0.05 g) and toluene (20 ml). The mixture was stirred at room temperature for 10 minutes, before addition of dicyclohexyl carbodiimide (0.75 g). This was then stirred at room temperature for 45 minutes.

More bis-succinyl-DMS-C15 (6.0 g at 6.4% in toluene=0.38 g) was added and the mixture stirred for a further 40 minutes. TLC showed only a faint trace for unreacted starting material.

The mixture was cooled in an ice bath for 15 minutes, then was filtered, and the solids washed with toluene (5 ml). The solution was used for flash chromatography. The best fractions were combined and evaporated down. The resulting green gum was dissolved in acetone (15 ml), filtered, and then evaporated down to give 1.6 g of a green oil which converted on standing to a pale green opaque soft solid. Approximate yield=77%

### Example 6

### (3-(4′-Methoxyphenyl),3-(4″-(succinylethoxy)phenyl)-6-morpholino-3H-naphtho[2,1-b]pyran)₂-DMS-C16

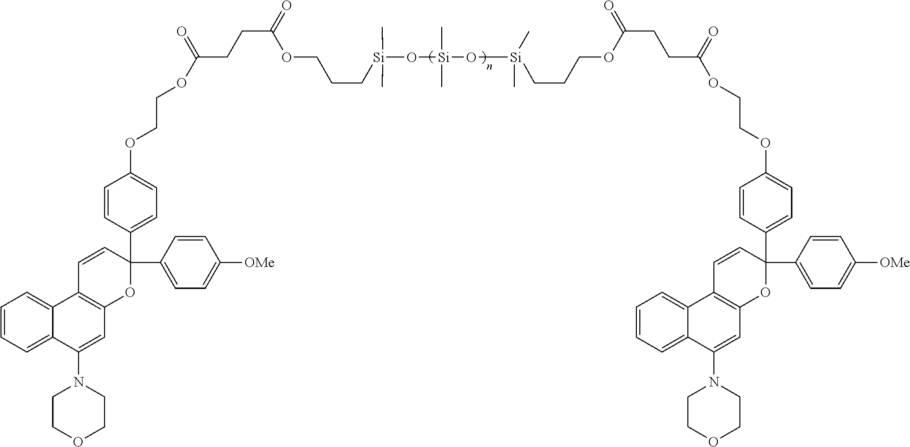

3-(4′-Methoxyphenyl),3-(4″-hydroxyethoxyphenyl)-6-morpholino-3H-naphtho[2,1-b]pyran (1.50 g) was mixed with a toluene solution of bis-succinyl-DMS-C16 (prepared according to Example 3, 14.7 g of 11.6% solution=1.71 g at 100%), toluene (20 ml) and dimethylaminopyridine (0.07 g). This was stirred for 2 minutes, then dicyclohexyl carbodiimide (0.67 g) was added and the mixture stirred at room temperature. The mixture became opaque after 1-2 minutes stirring.

After 45 minutes, thin layer chromatography (TLC) (3:1 petrol:acetone) indicated that some unreacted starting material remained. More of the toluene solution of bis-succinyl-DMS-C16 (6.1 g of 11.6% solution=0.71 g at 100%) was added, and the mixture stirred for 1 hour. At this point TLC indicated virtually no starting material remained.

The mixture was cooled to 4° C. for 45 minutes, then was filtered and the solids washed with toluene (5 ml). The solution was used for flash chromatography, eluting with a mixture of petroleum ether and ethyl acetate. This gave 2.7 g of an orange oil which hardened to an opaque orange solid. Yield=approximately 96%.

### Example 7

### (1,3-Dihydro-3,3-dimethyl-1-isobutyl-9′-succinyl-spiro[2H-indole-2,3′-3H-naphthol-2,1-b][1,4]oxazine)₂-DMS-C16

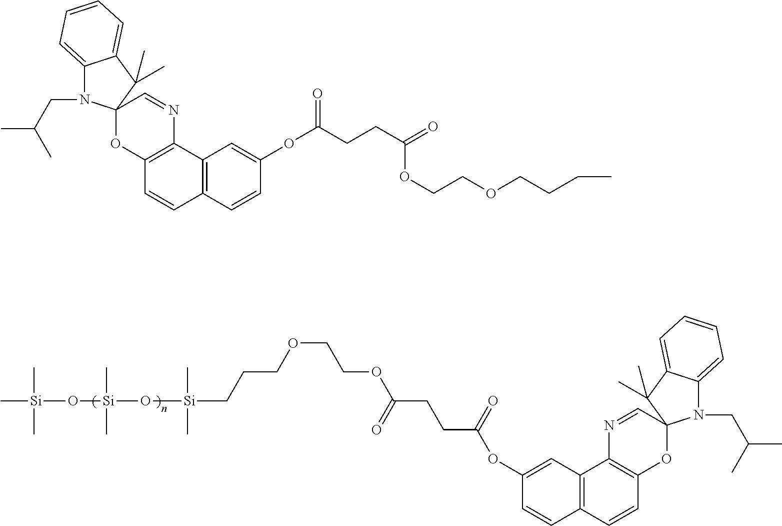

1,3-Dihydro-3,3-dimethyl-1-isobutyl-9′hydroxy-spiro[2H-indole-2,3′-3H-naphtho[2,1-b][1,4]oxazine] (1.50 g) was mixed with a toluene solution of bis-succinyl-DMS-C16 (prepared according to Example 3, 29.8 g of 9.7% toluene solution=2.90 g at 100%), toluene (20 ml) and dimethylamino pyridine (0.07 g). This was stirred for 2 minutes until all of the solid had dissolved. Dicyclohexyl carbodiimide (0.90 g) was added and the mixture stirred at room temperature for 45 minutes. After about 10 minutes, the solution became cloudy with the white precipitate of dicyclohexyl urea.

After 45 minutes, TLC (3:1 petrol:acetone) indicated that effectively all of the starting material had been converted to a less polar photochromic product. The mixture was cooled to 4° C. for 45 minutes, then was filtered and the solids washed with toluene (5 ml). The solution was used for flash chromatography, eluting with a mixture of petroleum ether and ethyl acetate. The best fractions were combined and evaporated down. The resulting blue oil was redissolved in acetone (30 ml), filtered and evaporated down again. This gave a pale blue-green oil: 2.3 g. Approximate yield=68%.

### Example 8

### (2-(4′-Pyrrolidinophenyl)-2-phenyl-5-phthaloylmethyl-6-anisyl-9-methoxy-2H-naphtho[1,2-b]pyran)₂-DMS-C15

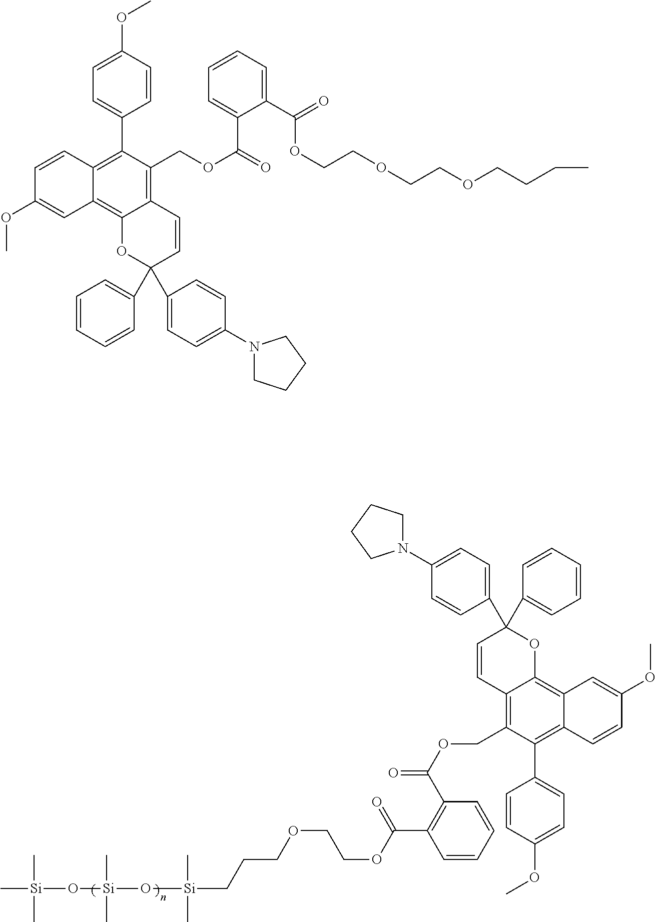

2-(4′-Pyrrolidinophenyl)-2-phenyl-5-hydroxymethyl-6-anisyl-9-methoxy-2H-naphtho[1,2-b]pyran (1.50 g) was mixed with a toluene solution of bis-phthaloyl-DMS-C15 (prepared according to Example 2, 22.8 g at 9.2%=2.10 g at 100%), toluene (20 ml), and dimethylaminopyridine (0.05 g). This was stirred for 2 minutes, then DCCI (0.60 g=1.10 mol/mol) was added. This was stirred for 45 minutes, at which point TLC showed that some unreacted starting material remained. More of the toluene solution of bis-phthaloyl-DMS-C15 (1.9 g of solution=0.17 g at 100%) was added and the mixture stirred for a further 40 minutes. TLC indicated that virtually no starting material remained, and so the mixture was cooled to 4° C. for 45 minutes. This was filtered and the solids washed with toluene (5 ml).

The solution was used for flash chromatography, eluting with a mixture of ethyl acetate and toluene. The best fractions were combined and evaporated down. The blue tar was dissolved in acetone (20 ml), filtered and evaporated down again to give 2.2 g of a dark blue tar. Approximate yield=69%.

### Example 9

### (2,2-Bis(4′-methoxyphenyl)-5-hydroxymethyl-6-methyl-2H-naphtho[1,2-b]pyran)₂-DMS-B12

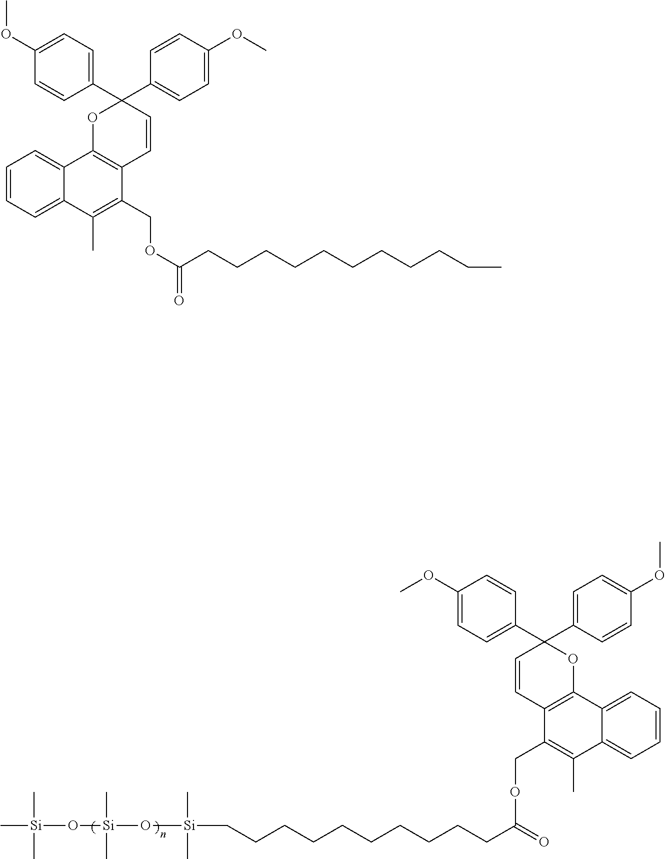

2,2-Bis(4′-methoxyphenyl)-5-hydroxymethyl-6-methyl-2H-naphtho[1,2-b]pyran (1.50 g) was mixed with the bis-carboxy-terminated siloxane DMS-B12 (2.10 g), dimethylaminopyridine (0.07 g) and toluene (35 ml) at room temperature. This was stirred for 2 minutes, then dicyclohexyl carbodiimide (0.80 g) was added and the mixture stirred for 45 minutes. TLC indicated that all of the starting material had been consumed. The mixture was cooled to 4° C. for 45 minutes. This was filtered and the solids washed with toluene (5 ml).

The solution was used for chromatography eluting with a mixture of petroleum ether and ethyl acetate. The best fractions were combined and evaporated down to give an orange-red oil: 1.3 g Approximate yield=41%.

### Example 10

### (2-(4′-Pyrrolidinophenyl)-2-phenyl-5-succinylmethyl-6-anisyl-9-methoxy-2H-naphtho[1,2-b]pyran)-DMS-C15-(3-phenyl-3-(4′-(succinylethoxy)phenyl)-6-morpholino-3H-naphtho[2,1-b]pyran)

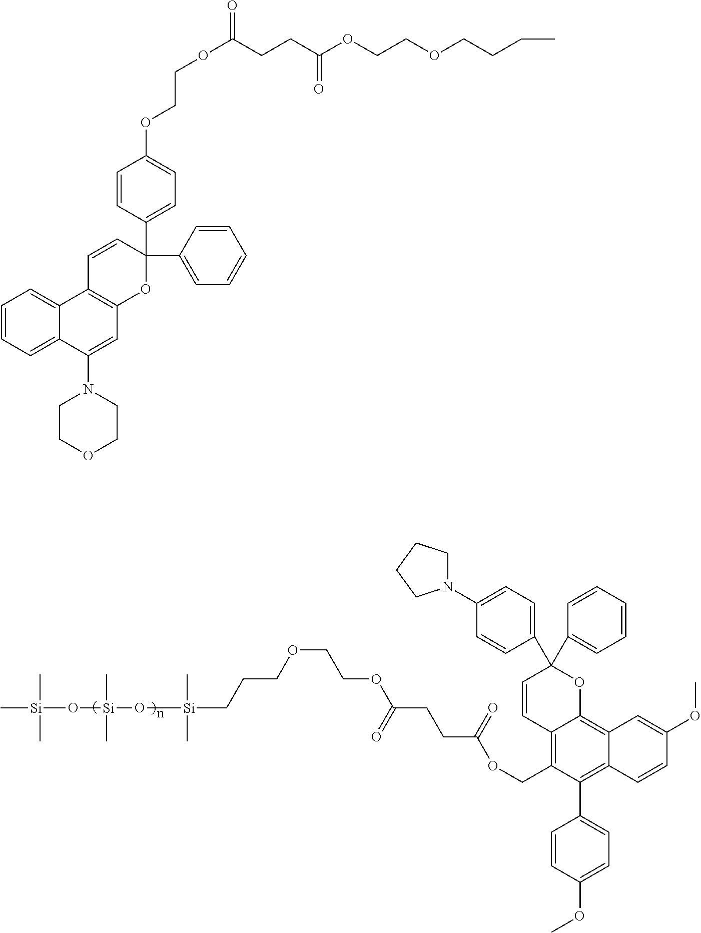

2-(4′-Pyrrolidinophenyl)-2-phenyl-5-hydroxymethyl-6-anisyl-9-methoxy-2H-naphtho[1,2-b]pyran (0.57 g=0.001 mol, blue-colouring pyran) was mixed with 3-phenyl-3-(4′-hydroxyethoxyphenyl)-6-morpholino-3H-naphtho[2,1-b]pyran (0.48 g=0.001 mol, yellow-colouring pyran), a toluene solution of bis-succinyl-DMS-C15 (prepared according to Example 1, 30.0 g at 6.8%=1.92 g at 100%) and dimethylaminopyridine (0.05 g) and stirred for 10 minutes at room temperature until all of the solid dissolved. Dicyclohexyl carbodiimide (0.90 g) was added, and the mixture stirred for 2 hours at room temperature. TLC (5:1 toluene:EtOAc) indicated that the two starting material photochromics had been consumed. The mixture was filtered to remove dicyclohexyl urea, which was washed with toluene (5 ml).

The solution was used for chromatography, eluting with a mixture of toluene and ethyl acetate. The first chromatography column removed most of the product with the blue-colouring pyran at each end of the chain, and most of the product with the yellow-colouring pyran at each end of the chain, with the remaining fractions containing mostly the required “mixed” product. These fractions were combined, evaporated down and chromatographed again. The best fractions were combined, evaporated down, dissolved in acetone (20 ml), filtered, and evaporated down again to give a viscous yellow-brown oil: 1.15 g. Approximate yield=52%.

### Example 11

### (1,3-Dihydro-3,3-dimethyl-1-neopentyl-9′-succinyl-spiro[2H-indole-2,3′-3H-naphtho[2,1-b][1,4]oxazine])₂-DMS-A214

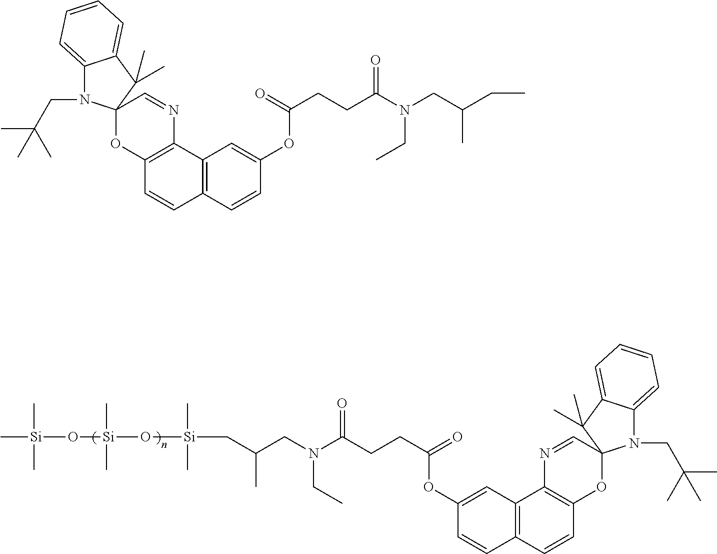

1,3-Dihydro-3,3-dimethyl-1-neopentyl-9′-hydroxy-spiro[2H-indole-2,3′-3H-naphtho[2,1-b][1,4]oxazine] (0.47 g) was mixed with bis-Succinamido-DMS-A214 (12.5 g of 17.1% toluene solution=2.14 g at 100%). Dimethylaminopyridine (0.03 g) was added and the mixture stirred for 1 minute before addition of dicyclohexyl carbodiimide (0.29 g). The mixture was stirred for 45 minutes and TLC indicated a non-polar product smear, and no spot for unreacted starting material.

The mixture was cooled in an ice bath for 45 minutes, then was filtered to remove dicyclohexyl urea. The solution was used for chromatography, eluting with ethyl acetate and petroleum ether. The best fractions were combined and evaporated down. The resulting green-brown oil was dissolved in acetone (30 ml), filtered and then evaporated down again to give 1.8 g=82%.

### Example 12

### (2,2-Bis(4∝-methoxyphenyl)-5-succinylmethyl-6-methyl-2H-naphtho[1,2-b]pyran)₂-DMS-C15

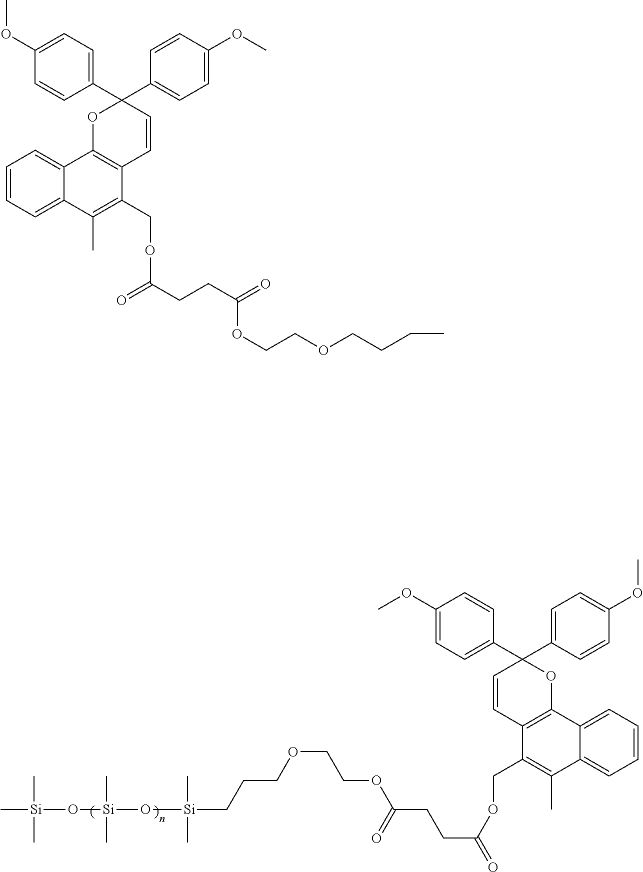

2,2-Bis(4′-methoxyphenyl)-5-hydroxymethyl-6-methyl-2H-naphtho[1,2-b]pyran was mixed with bis-succinyl-DMS-C15 (2.0 g), toluene (20 ml) and dimethylaminopyridine (0.05 g). This was stirred for 2 minutes, then dicyclohexyl carbodiimide (0.51 g) was added. The mixture was stirred for 45 minutes and TLC (3:1 toluene:EtOAc) indicated a main spot for polydialkylsiloxane-bridged bi-photochromic product, with effectively no starting material remaining. The mixture was cooled in an ice bath for 1 hour, then was filtered and the dicyclohexyl urea washed with toluene (5 ml).

The solution was used for chromatography, eluting with ethyl acetate and petroleum ether. The best fractions were combined and evaporated down to give a dark orange oil. This was dissolved in acetone (approx 40 ml) and filtered. The solution was evaporated down to give: 2.1 g. Approximate yield 90%.

### Comparative Compounds

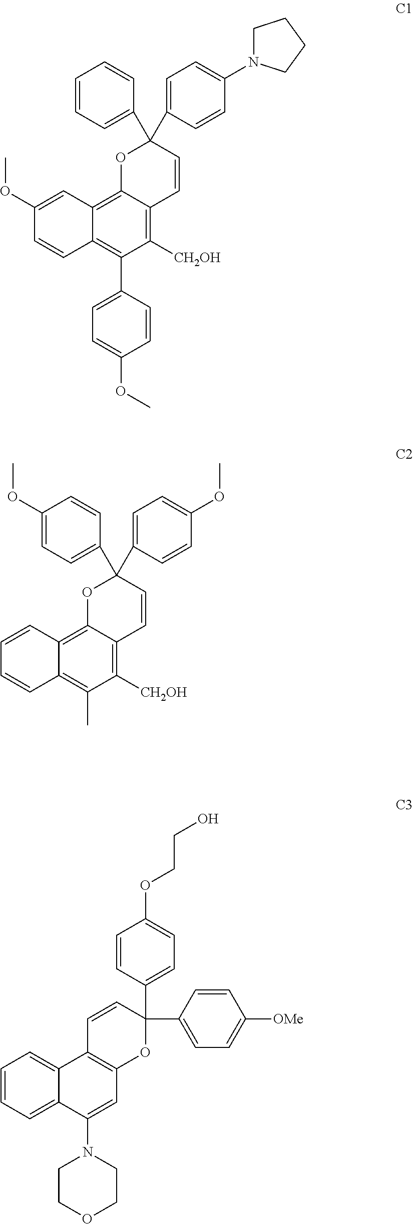

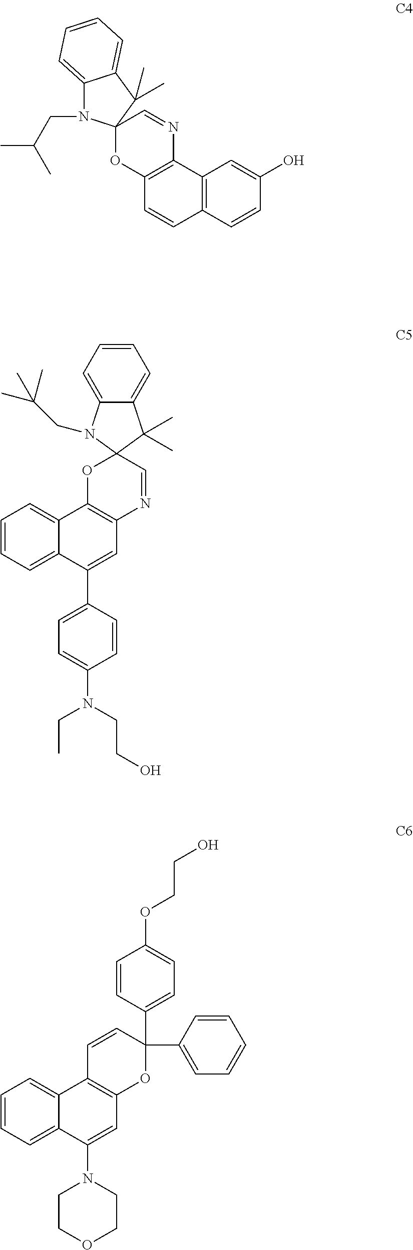

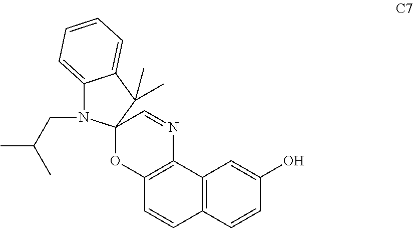

### Testing for Fade Speed and Intensity in Acrylate-Based Lenses

Samples of the Examples 5 to 11 and the comparative compounds C1 to C6 were dissolved in ethoxylated(4)bisphenol A dimethacrylate monomer at 250 ppm by weight, and then cured at 200° C. in lens moulds. The resulting lenses were allowed to cool and stand for at least 24 hours before testing. The lenses were activated for 10 minutes in a constant temperature water bath at 23° C., with a 50 Klux light source filtered to Air Mass 2 standard. The resulting induced absorption (Delta Abs) was measured at lambda max of the compound. The light source was turned off and the resulting fade was monitored, giving the time to fade to half the initial absorption (T_{1/2}) and to one quarter of the initial absorption (T_{3/4}).

The parameter “Adjusted Delta Abs” allows for the molecular weight of the polydialkylsiloxane-bridged bi-photochromic compound, the molecular weight of the unbridged comparative compound and the number of photochromic units present. This is calculated as follows:

Adjusted Delta Abs=(Delta Abs Example compound×(Mol Wt example compound)/(Mol Wt comparative compound))/Number of photochromic units present in Example compound

For Example 10, which has a different photochromic unit at each end of the chain, the absorptions from each photochromic unit are treated separately.

| | L max | L max | T½ | | Delta | Adjusted |
|---|---|---|---|---|---|---|
| Compound | 1 (nm) | 2 (nm) | (sec) | T¾ (sec) | Abs | Delta Abs |
| Example 5 | 635 | | 47 | 106 | 0.605 | 1.249 |
| C5 | 635 | | 193 | 257 | 1.223 | 1.223 |
| Example 6 | 455 | | 40 | 104 | 0.802 | 2.336 |
| C3 | 455 | | 82 | 335 | 0.879 | 0.879 |
| Example 8 | 590 | | 58 | 216 | 0.338 | 0.714 |
| C1 | 585 | | 197 | 1432 | 0.411 | 0.411 |
| Example 9 | 495 | | 68 | 205 | 0.541 | 0.877 |
| C2 | 500 | | 104 | 512 | 0.583 | 0.583 |
| Example 10 | 440 | | 54 | 162 | 0.410 | 1.893 |
| C6 | 440 | | 137 | 526 | 1.282 | 1.282 |
| Example 10 | | 575 | 75 | 199 | 0.180 | 0.700 |
| C1 | | 585 | 197 | 1432 | 0.411 | 0.411 |
| Example 11 | 610 | | 23 | 60 | 0.0773 | 0.3589 |
| C7 | 610 | | 62 | 230 | 0.4809 | 0.4809 |

As can be seen from the above table, the T_{1/2} values for the tailed dimers are between 29.4% and 65.4% of the T_{1/2} values of the corresponding comparative compounds. The T_{3/4} values of the tailed dimers show even greater improvements, being between 13.9% and 40.0% of the T_{3/4} values of the corresponding comparative compounds.

The values for Adjusted Delta Abs indicate that the colour strengths of the polydialkylsiloxane-bridged bi-photochromic compound range from slightly weaker than the comparative compounds (Example 11) to considerably stronger (Examples 6, 8, 9 and 10).

### Heat Stability Testing

Samples of compounds of Example 7 and Example 12 and the corresponding comparative compounds C4 and C2 were incorporated at 250 ppm into polycarbonate, and polystyrene at different processing temperatures using a Boy 35M injection moulding machine, giving rectangular chips. The chips were measured for absorption using the same equipment as was used for measuring lenses. The chips were measured for yellowness index (as ASTM D1925) using a Datacolor Spectraflash SF450 colour spectrometer.

### (i) Example 12 and C2 Incorporated at 250 ppm in Polycarbonate, Processed at 315° C. and 330° C.

| | | | | Ratio | Yellow- |
|---|---|---|---|---|---|
| | Initial Abs | Delta Abs | Adjusted Delta | Adjusted | ness |
| | 315° C. | 315° C. | Abs 315° C. | Delta Abs | index |
| C2 | 0.0834 | 0.1282 | 0.1282 | | 28.0 |
| Example | 0.0497 | 0.1556 | 0.3623 | 2.83:1 | 22.2 |
| 12 | | | | | |
| | | | | Ratio | Yellow- |
| | Initial Abs | Delta Abs | Adjusted Delta | Adjusted | ness |
| | 330° C. | 330° C. | Abs 330° C. | Delta Abs | index |
| C2 | 0.1097 | 0.0699 | 0.0699 | | 36.2 |
| Example | 0.0630 | 0.0984 | 0.2292 | 3.28:1 | 26.6 |
| 12 | | | | | |

### (ii) Example 7 and C4 Incorporated at 250 ppm in Polystyrene.

| | | | | Ratio | Yellow- |
|---|---|---|---|---|---|
| | Initial Abs | Delta Abs | Adjusted Delta | Adjusted | ness |
| | 230° C. | 230° C. | Abs 230° C. | Delta Abs | index |
| C4 | 0.0227 | 0.0832 | 0.0832 | | 14.7 |
| Example 7 | 0.02467 | 0.0892 | 0.1924 | 2.31 | 9.9 |
| | | | | Ratio | Yellow- |
| | Initial Abs | Delta Abs | Adjusted Delta | Adjusted | ness |
| | 250° C. | 250° C. | Abs 250° C. | Delta Abs | index |
| C4 | 0.0658 | 0.0831 | 0.0831 | | 19.3 |
| Example 7 | 0.01742 | 0.0924 | 0.1995 | 2.40 | 10.8 |

1-25. (canceled)

26. A bi-photochromic molecule comprising two photochromic moieties linked via a polydialkylsiloxane (PDAS) oligomer, and having the general formula
PC-L-PDAS-L′-PC′
wherein PC and PC′, which may be the same or different, represent photochromic moieties of general structure I to IV, 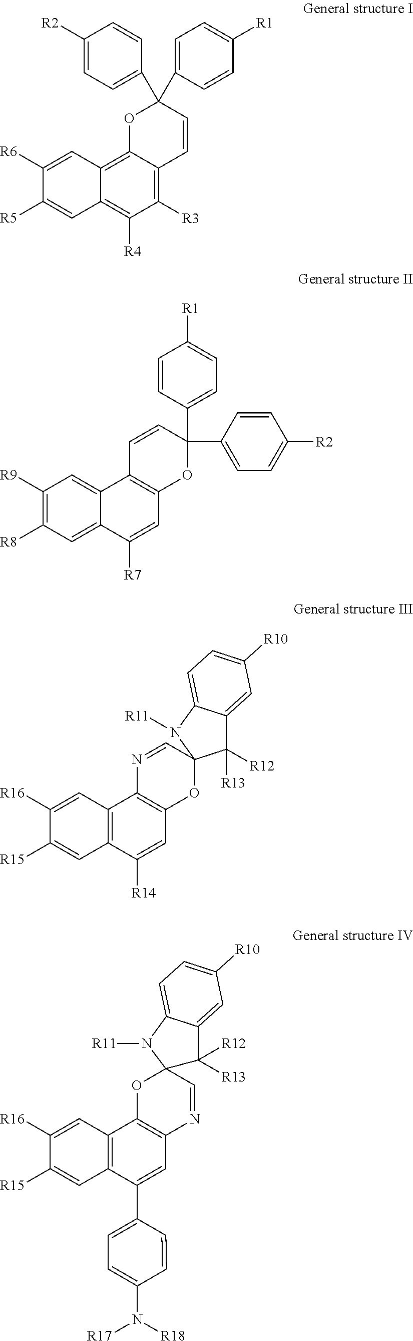
wherein R1 and R2 independently represent hydrogen, linear or branched C₁₋₁₀ alkyl, linear or branched C₁₋₁₀ alkoxy, C₁₋₁₀ hydroxyalkoxy, C₁₋₁₀ alkoxy(C₁₋₁₀)alkoxy, phenyl, C₁₋₁₀ alkoxyphenyl, halogen, C₁₋₅ haloalkyl, C₁₋₅ alkylamino, C₁₋₅ dialkylamino, arylamino, diarylamino, aryl C₁₋₅ alkylamino, or a cyclic amino group;
R3 represents hydrogen, linear or branched C₁₋₁₀ alkyl, up to C₂₀ cycloalkyl, up to C₂₀ bicycloalkyl, linear or branched C₂₋₁₀ alkenyl, linear or branched C₁₋₁₀ alkoxy, C₁₋₁₀ hydroxyalkyl, C₁₋₁₀ alkoxy(C₁₋₁₀)alkyl, C₁₋₁₀ aminoalkyl, linear or branched C₁₋₂₀ alkoxycarbonyl, carboxyl, halogen, aryloxycarbonyl, formyl, acetyl or aroyl;
R4 represents, phenyl, C₁₋₁₀ alkoxyphenyl, C₁₋₁₀ dialkoxyphenyl, C₁₋₁₀ alkylphenyl, C₁₋₁₀ dialkylphenyl or one of the groups specified for R3;
or R3 and R4 together form a cyclic structure of the type 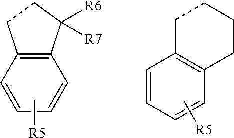
R5, R6, R7, R8, R9, R10, R14, R15, R16 are as defined for R1 and R2;
R11 represents linear or branched C₁₋₂₀ alkyl, C₃₋₂₀ cycloalkyl, C₆₋₂₀ bicycloalkyl, (C₁₋₅ alkyl)aryl, (C₁₋₅ alkyl)cycloalkyl, (C₁₋₅ alkyl)bicycloalkyl, C₁₋₅ haloalkyl, C₁₋₅ dihaloalkyl, or C₁₋₅ trihaloalkyl;
R12 and R13 represent C₁₋₁₀ alkyl, C₁₋₅ alkyl alkoxycarbonyl, or together form a C₅₋₇ ring;
R17 and R18 represent linear or branched C₁₋₁₀ alkyl, C₁₋₁₀ hydroxyalkyl, or together form a C₅₋₇ ring;
L and L′ which may be the same or different, represent a linking group.

27. A bi-photochromic molecule according to claim 26 wherein the polydialkylsiloxane (PDAS) oligomer is of the formula: 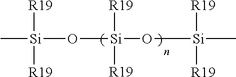
wherein R19 is C₁₋₁₀ alkyl, and n is an integer of from 4 to 75 inclusively.

28. A bi-photochromic molecule according to claim 26 wherein the polydialkylsiloxane oligomer is a polydimethylsiloxane oligomer.

29. A bi-photochromic molecule according to claim 26, wherein L and L′ represent a linking group of the form; 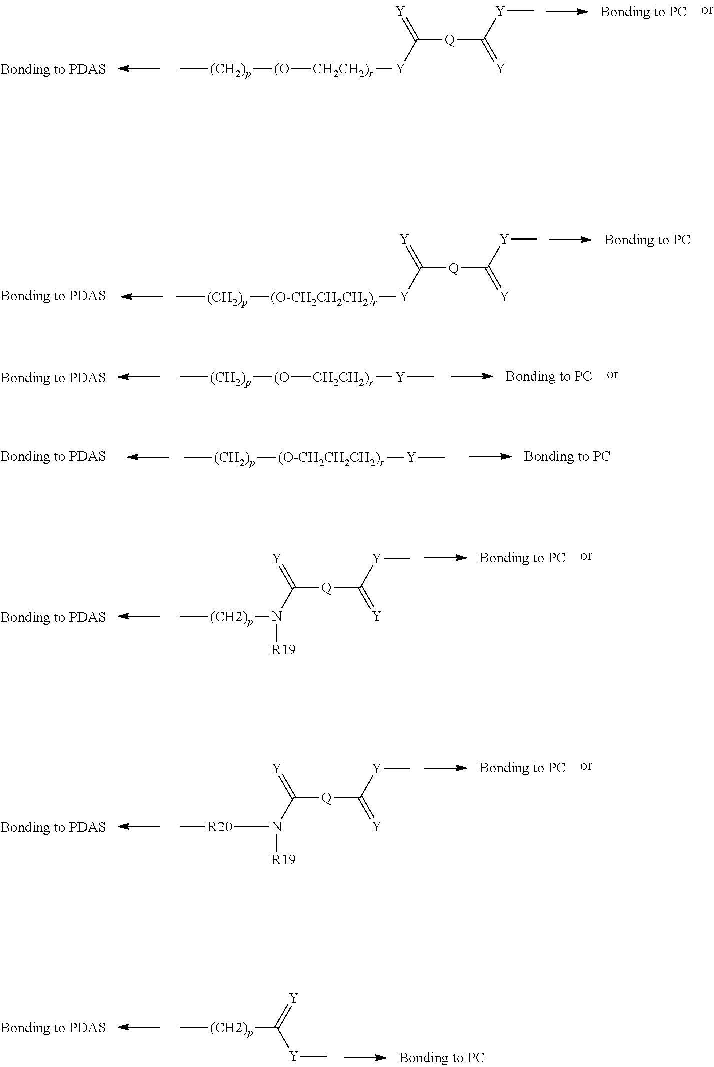
wherein Y is independently oxygen or sulphur, R19 is hydrogen or C₁₋₁₀ linear or branched alkyl, R20 is C₁₋₁₀ linear or branched alkyl, p is an integer from 1 to 15, and r is an integer from 0 to 10, and wherein Q is linear or branched C₁₋₁₀ alkyl, C₁₋₁₀ alkenyl or 1,2-, 1,3, or 1,4-substituted aryl, or substituted heteroaryl.

30. A bi-photochromic molecule according to claim 28 wherein the polydialkylsiloxane oligomer is selected from DMS-B12, DMS-C15, DMS-C16, DMS-C21, DMS-A11, DMS-A12, DMS-A15, DMS-A21, DMS-A211, DMS-A214, KF-6001, KF-6002, KF-6003, KF-8010, X-22-160AS, X-22-162A, X-22-161A, X-22-161B, X-22-162C, and Silaplane FM-44, the structures of which are shown below:

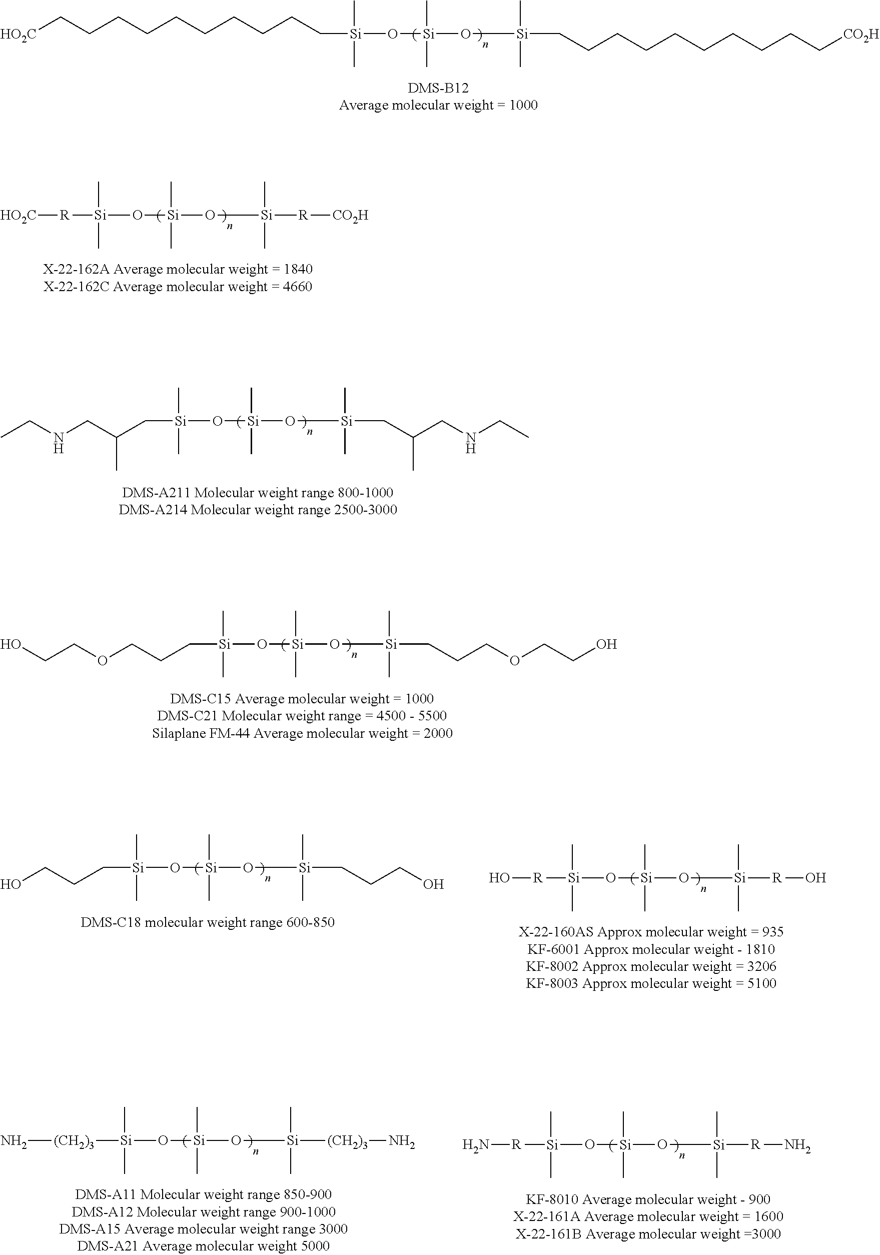

31. A bi-photochromic molecule according to claim 29 wherein each of PC and PC′ is either a naphtho[1,2-b]pyran of general structure 1 or a naphtho[2,1-b]pyran of general structure 2.

32. A bi-photochromic molecule according to claim 31 wherein both PC and PC′ are a naphtho[1,2-b]pyran of general structure 1.

33. A bi-photochromic molecule according to claim 31 wherein both PC and PC′ are a naphtho[2,1-b]pyran of general structure 2.

34. A bi-photochromic molecule according to claim 31 wherein one of PC and PC′ is a naphtho[1,2-b]pyran of general structure 1, and the other is a naphtho[2,1-b]pyran of general structure 2.

35. A bi-photochromic molecule according to claim 29 wherein Y represents oxygen, Q represents —(CH₂CH₂)—, and R19 is methyl.

36. A bi-photochromic molecule according to claim 29 wherein Y 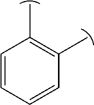
represents oxygen, Q represents
and R19 is methyl.

37. A bi-photochromic molecule according to claim 29 which is (1,3-dihydro-3,3-dimethyl-1-neopentyl-6′-(4″-N-ethyl, N-(succinylethyl)anilino)spiro[2H-indole-2,3′-3H-naphtho[1,2-b][1,4]oxazine)₂-DMS-C15, where DMS-C15 is as defined above.

38. A bi-photochromic molecule according to claim 29 which is (3-(4′-methoxyphenyl),3-(4″-(succinylethoxy)phenyl)-6-morpholino-3H-naphtho[2,1-b]pyran)₂-DMS-C16, where DMS-C16 is as defined above.

39. A bi-photochromic molecule according to claim 29 which is (3-(4′-methoxyphenyl),3-(4″-(succinylethoxy)phenyl)-6-morpholino-3H-naphtho[2,1-b]pyran)₂-DMS-C15 where DMS-C15 is as defined above.

40. A bi-photochromic molecule according to claim 29 which is (1,3-dihydro-3,3-dimethyl-1-isobutyl-9′-succinyl-spiro[2H-indole-2,3′-3H-naphtho[2,1-b][1,4]oxazine)₂-DMS-C16 where DMS-C16 is as defined above.

41. A bi-photochromic molecule according to claim 29 which is (2-(4′-pyrrolidinophenyl)-2-phenyl-5-phthaloylmethyl-6-anisyl-9-methoxy-2H-naphtho[1,2-b]pyran)₂-DMS-C15, where DMS-C15 is as defined above.

42. A bi-photochromic molecule according to claim 29 which is (2,2-bis(4′-methoxyphenyl)-5-hydroxymethyl-6-methyl-2H-naphtho[1,2-b]pyran)₂-DMS-B12, where DMS-B12 is as defined above.

43. A bi-photochromic molecule according to claim 29 which is (2-(4′-pyrrolidinophenyl)-2-phenyl-5-succinylmethyl-6-anisyl-9-methoxy-2H-naphtho[1,2-b]pyren)-DMS-C15-(3-phenyl-3-(4′-(succinylethoxy)phenyl)-6-morpholino-3H-naphtho[2,1-b]pyran), where DMS-C15 is as defined above.

44. A bi-photochromic molecule according to claim 29 which is (1,3-dihydro-3,3-dimethyl-1-neopentyl-9′-succinyl-spiro[2H-indole 2,3′-3H-naphtho[2,1-b][1,4]oxazine])₂-DMS-A214, where DMS-A214 is as defined above.

45. A bi-photochromic molecule according to claim 29 which is (2,2-Bis(4′-methoxyphenyl)-5-succinylmethyl-6-methyl-2H-naphtho[1,2-b]pyran)₂-DMS-C15, where DMS-C15 is as defined above.

46. An ophthalmic lens comprising a bi-photochromic molecule according to claim 26.

47. A polymeric host material comprising a bi-photochromic molecule according to claim 26.

48. A method of manufacturing a bi-photochromic molecule as defined in claim 26, comprising reacting a polydialkylsiloxane oligomer, linking group, and one or more photochromic compounds to form the bi-photochromic molecule.

49. A method according to claim 48 wherein the linking group is attached to the polydialkylsiloxane oligomer prior to reaction with the one or more photochromic compounds.

50. A method according to claim 48 wherein the linking group is attached to the one or more photochromic compounds prior to reaction with the polydialkylsiloxane oligomer.
